(19) United States
(12) Patent Application Publication
(10) Pub. No.: US 2011/0245568 A1
(43) Pub. Date: Oct. 6, 2011

(54) Dehydrogenation Reactions of N-Butene to Butadiene

(75) Inventors: Olga Khabashesku, Houston, TX (US); James R. Butler, League City, TX (US); Dariusz Wachowicz, Friendswood, TX (US)

(73) Assignee: Fina Technology, Inc., Houston, TX (US)

(21) Appl. No.: 13/094,877

(22) Filed: Apr. 27, 2011

Related U.S. Application Data
(63) Continuation-in-part of application No. 12/506,317, filed on Jul. 21, 2009.
Application No. 12/506,317 is a continuation-in-part of application No. 12/177,740, filed on Jul. 22, 2008.

(51) Int. Cl. C07C 5/03

(52) U.S. Cl. 585/629; 585/627

(57) ABSTRACT

A method for the dehydrogenation of n-butene to form butadiene over a dehydrogenation catalyst with a butadiene yield of at least 40 mol % is disclosed. Embodiments involve operating the dehydrogenation reactor at a pressure of 1,000 mbar or less.

### CROSS-REFERENCE TO RELATED APPLICATIONS

This application is a continuation-in-part of U.S. patent application Ser. No. 12/506,317 filed Jul. 21, 2009 which is a continuation-in-part of U.S. patent application Ser. No. 12/177,740 filed on Jul. 22, 2008.

### FIELD

The present invention generally relates to the dehydrogenation of hydrocarbons.

### BACKGROUND

Butadiene, also known as 1,3-butadiene, is a common monomer in the production of synthetic rubber. Butadiene is a raw material for many high-volume industrial applications including tire manufacturing.

Butadiene is most commonly produced as a by-product in the steam cracking processes used to produce ethylene and other olefins. According to this process, the crude C4 stream isolated from the steam cracking is fed to butadiene extraction units, where butadiene is separated from the other C4s by extractive distillation. The amount of crude C4s produced in steam cracking is dependent on the composition of the feed to the cracking unit. Heavier feeds, such as naphtha, yield higher amounts of C4s and butadiene than do lighter feeds. Crackers using light feeds typically produce low quantities of C4s and do not have butadiene extraction units. Ethylene production from lighter feeds, such as C2, C3 and C4s, yield significantly less amounts of butadiene. As well, emerging technologies for producing ethylene and propylene such as Methanol-To-Olefins (MTO), metathesis and olefins catalytic cracking generally do not co-produce butadiene. Production of on-purpose butadiene may become necessary.

It may be desirable to utilize equipment that has the capability of producing more than a single product. For example, it may be beneficial to have the ability to utilize equipment typically used for the dehydrogenation of ethylbenzene to styrene also for the dehydrogenation of other hydrocarbons. It may also be desirable to utilize commercial catalysts that are typically used for dehydrogenation reactions such as ethylbenzene to styrene reactions for the dehydrogenation of other hydrocarbons to more unsaturated hydrocarbons, such as the dehydrogenation of olefins to form diolefins.

Commercial catalysts for the dehydrogenation of ethylbenzene may be useful in the dehydrogenation of other hydrocarbons. However, they may require high steam-to-hydrocarbon ratios, resulting in relatively short catalyst life. The higher steam-to-hydrocarbon ratio will increase the operating cost due to the need for more steam, therefore having an adverse effect on the economics of the process. Further, due to the decrease of catalyst activity, steaming of the catalyst is required in a regeneration step to restore activity. The operation of steaming the catalyst has a detrimental economic effect from the increased steam required and the reduction in product produced during this regeneration operation. The repeated action of steaming the catalyst typically results in a decrease in the useful life of the catalyst.

Further, dehydrogenation of olefins can result in undesirable side products including acetylenic compounds.

It may be desirable to be able to utilize equipment and catalysts typically used to dehydrogenate ethylbenzene to styrene also for the dehydrogenation of hydrocarbons, in a method that exhibits increased catalyst life with a reduction in the need for catalyst steaming, and that does not result in a significant amount of undesirable side products like acetylenic compounds.

### SUMMARY

The present invention in its many embodiments is a method for the dehydrogenation of n-butene over a dehydrogenation catalyst, at a pressure of 1,000 mbar or less, under reaction conditions to produce 1,3-butadiene at a yield level of at least 40 mol %. The yield optionally can be at least 45 mol %.

In an embodiment, either by itself or in combination with any other embodiment, steam can be supplied in a steam to hydrocarbon molar ratio of at least 10:1, optionally between 10:1 and 30:1.

In an embodiment, either by itself or in combination with any other embodiment, the dehydrogenation reaction can be operated in a reactor at a LHSV of from 0.1 hr⁻¹ to 1.0 hr⁻¹.

In an embodiment, either by itself or in combination with any other embodiment, the dehydrogenation reaction can be operated in a reactor at a pressure of 350 mbar or less, optionally 300 mbar or less.

In an embodiment, either by itself or in combination with any other embodiment, the dehydrogenation reaction can be operated in a reactor at a temperature of at least 500° C., optionally at least 600° C.

In an embodiment, either by itself or in combination with any other embodiment, the temperature can be increased in order to keep the 1,3-butadiene molar yield at least 40 mol %, optionally at least 45 mol %.

In an embodiment, either by itself or in combination with any other embodiment, the dehydrogenation catalyst can be a catalyst that is used for the dehydrogenation of ethylbenzene to styrene.

In an embodiment, either by itself or in combination with any other embodiment, the dehydrogenation catalyst can have an average effective pore diameter of at least 500 nanometers. The dehydrogenation catalyst can have ferric oxide and potassium as components. In an embodiment, the dehydrogenation catalyst is a commercial catalyst.

In an embodiment, the dehydrogenation reaction produces less than 1 mol % of undesirable acetylenic side products. In an embodiment, the dehydrogenation reaction can operate in excess of 30 days, optionally 45 days, before the catalyst becomes a deactivated catalyst.

Other possible embodiments include two or more of the above embodiments of the invention. In an embodiment the method includes all of the above embodiments and the various procedures can be carried out in any order.

### BRIEF DESCRIPTION OF DRAWINGS

FIG. 1 shows the molar yield of butadiene and reactor inlet temperature over catalyst age for the dehydrogenation reaction of the example.

FIG. 2 shows the 1-butene conversion and reactor inlet temperature over catalyst age for the dehydrogenation reaction of the example.

### DETAILED DESCRIPTION

The present invention involves the production of diolefins by dehydrogenating an olefin containing feed. Specifically, the present invention is for production of butadiene by dehydrogenating an n-butene containing feed. The feed is subjected to catalytic dehydrogenation under vacuum conditions that enable the dehydrogenation of the n-butene to form a product having a 1,3-butadiene content equivalent to a yield of at least 40 mol %.

Equation 1 shows the reactions that take place to convert 1-butene into 1,3-butadiene. The first stage of 1-butene dehydrogenation is isomerisation, in which 1-butene changes into 2-butene isomers. Conversion of 1-butene into 2-butene is thermodynamically favorable because of the stabilizing effect of two alkyl groups on either side of the olefin π-bond. The second stage is dehydrogenation, in which 1,3-butadiene is formed along with hydrogen gas. Dehydrogenation is highly endothermic, and high temperatures around 600° C. can be economical for the conversion of n-butene to 1,3-butadiene. Suitable reaction temperature for the invention can range from 300° C. to 800° C., or from 500° C. to 650° C.

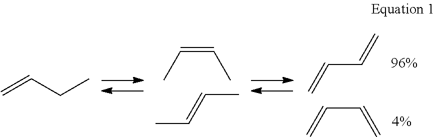

Steam can be added to the dehydrogenation reactor to aid the reaction. Because dehydrogenation involves an increase in the number of moles of gas produced, the reaction can be favored by the addition of steam to reduce partial pressure. Steam can also reduce coke deposition, by reacting with carbon to form carbon monoxide and hydrogen gas. Reducing coke formation can prolong catalyst life and reduce the need for frequent regeneration.

In embodiments of the dehydrogenation reaction, steam and the n-butene containing hydrocarbon feedstock can be supplied in a steam to hydrocarbon molar ratio of between 1:1 to 30:1, optionally between 10:1 to 25:1; optionally between 20:1 to 25:1. The steam can be mixed with the hydrocarbon either prior to introduction to the reactor, or the steam and hydrocarbon can be supplied separately to the reactor through separate lines. The steam is condensed and forms a liquid portion, this liquid water along with any liquid hydrocarbons that may have been present in the feed or produced in the reaction, such as aromatics, for example benzene, toluene or xylene, can be drained from the reactor or a subsequent separation stage, in any suitable method. The reacted hydrocarbon can be removed as either a liquid or a vapor, depending on the reactor conditions.

Under the conditions of the present invention, substantially all of the produced butadiene and unreacted hydrocarbon containing feed are vaporized and are removed in a vapor phase by any suitable method, such as a vacuum compressor, which can maintain the reactor pressure at the desired vacuum conditions.

In an embodiment there are one or more reactors, in parallel or series, wherein the catalyst is located and one or more reaction zones exist. In addition to the reactor, there may be a subsequent separation stage that enables the liquid from the reactor to be recovered and the vapor product to be removed. A heat exchanger may also be utilized to cool the reaction effluent prior to the separation stage. The operating pressure of a separation stage may be essentially the same as the outlet pressure of the reactor, other than the pressure drop that may occur across the heat exchanger. In alternate embodiments the operating pressure of a separation stage may be different than the reactor.

In an embodiment a dehydrogenation reactor can be modified to enable the removal of a vapor stream from the reactor and reduce the reactor pressure to vacuum conditions of 1000 mbar or less, optionally 500 mbar or less, optionally 350 mbar or less. Methods and processes of dehydrogenation disclosed in U.S. patent application Ser. No. 11/811,084 filed Jun. 8, 2007 by Merrill, incorporated by reference herein, may be suitable for embodiments of the present invention.

The dehydrogenation catalyst can be any dehydrogenation catalyst having a large enough pore size in order to avoid excessive diffusion limitations leading to restriction of the conversion of n-butene to butadiene, such as for a non-limiting example, those with an average effective pore diameter of at least 300 nanometers, at least 400 nanometers, or at least 500 nanometers. Subject to the pore diameter restrictions, the dehydrogenation catalyst may be of any suitable type, such as a catalyst containing iron as a major component with a lesser amount of potassium.

In a particular application of the invention the dehydrogenation catalyst is a ferric oxide, potassium carbonate based dehydrogenation catalyst having a relatively large average pore diameter, such as a pore diameter of at least 500 nanometers. Suitable catalyst compositions may comprise ferric oxide in amounts ranging from 40 to 80 wt %, potassium oxide or potassium carbonate in an amount of about 5 to 30 wt % and may also include lesser amounts of cerium, and other suitable catalyst promoters, such as from 0.1 wt % to 5 wt %. Catalysts disclosed in U.S. patent application Ser. No. 11/811,084 filed Jun. 8, 2007 by Merrill, incorporated by reference herein, may be utilized in the present invention.

In an aspect, the catalyst may be formed by milling the iron and potassium components with, for example, a plastic hydraulic cement binder followed by extruding the material to form catalyst particles of about from 2.5 mm to 5.0 mm in diameter having an average effective pore diameter of at least 500 nanometers. More specifically the dehydrogenation catalyst may have an average effective pore diameter of at least 500 nanometers and may have an average effective pore diameter of between 500 nanometers and 2,000 nanometers, optionally between 500 nanometers and 1,500 nanometers, optionally between 500 nanometers and 1,000 nanometers.

The catalyst can be a dehydrogenation catalyst used in the production of styrene from ethylbenzene. Such a catalyst may also be used for other dehydrogenation reactions, including the dehydrogenation of C5 alkenes and isoamylene. The dehydrogenation catalyst can be a commercial dehydrogenation catalyst, for example Hypercat sold by CRI Catalyst, which is a non-chromium-containing iron oxide catalyst used for the dehydrogenation of ethylbenzene to styrene. The dehydrogenation catalyst can also be, by non-limiting example: Styromax Plus from Sud-Chemie or Hypercat GV from Criterion.

In the present invention the LHSV can be any flow rate wherein the subject reaction can be achieved; such as for example embodiments of the invention can range from 0.1 hr⁻¹ to 10.0 hr⁻¹, or from 0.1 hr⁻¹ to 5.0 hr⁻¹, or from 0.1 hr⁻¹ to 1.0 hr⁻¹.

By operating under reduced pressure equilibrium will be shifted towards butadiene. Suitable reactor pressure for the invention can be less than 1000 mbar, optionally can range from 100 mbar to 1000 mbar, or from 200 mbar to 900 mbar, or optionally from 200 mbar to 500 mbar. In an embodiment the reactor pressure is operated at less than 350 mbar. Reduced pressures can also prolong catalyst life.

The product butadiene may be used in the production of synthetic rubbers, such as copolymers containing polystyrene, for example.

An experimental example is now provided, which is intended to provide a better understanding of the present invention and is not intended to limit the scope of the invention in any way.

### EXAMPLE

A 1-butene containing hydrocarbon stream was converted to butadiene over a commercial catalyst, Hypercat, from CRI catalyst. The reactor was a dehydrogenation reactor that had been used previously for the dehydrogenation of isoamylene to isoprene. Around 500 mL of Hypercat was loaded in the reactor at low temperature. After a pressure check, the reactor was brought to 300° C. with a nitrogen flow. Steam flow was started at 531 g/hr of H₂O and the temperature was raised to 500° C. Nitrogen flow was stopped and isoamylene was started at 105 g/hr. The temperature was increased overnight to 600° C., and the pressure was decreased to 300 mbar. The temperature was adjusted to achieve 40% isoprene yield in the effluent. When yield of isoprene reached 45 mol %, the feed was then switched from isoamylene to n-butene. Samples were collected daily, using a chilling bath at −78° C., and the off-gas flow rate was measured utilizing a wet test meter installed in the fume hood. LHSV was 0.31 hr⁻¹, and the steam to hydrocarbon ratio was from 20:1 to 24:1. The temperature was adjusted until a steady production of butadiene above 40 mol % conversion was achieved.

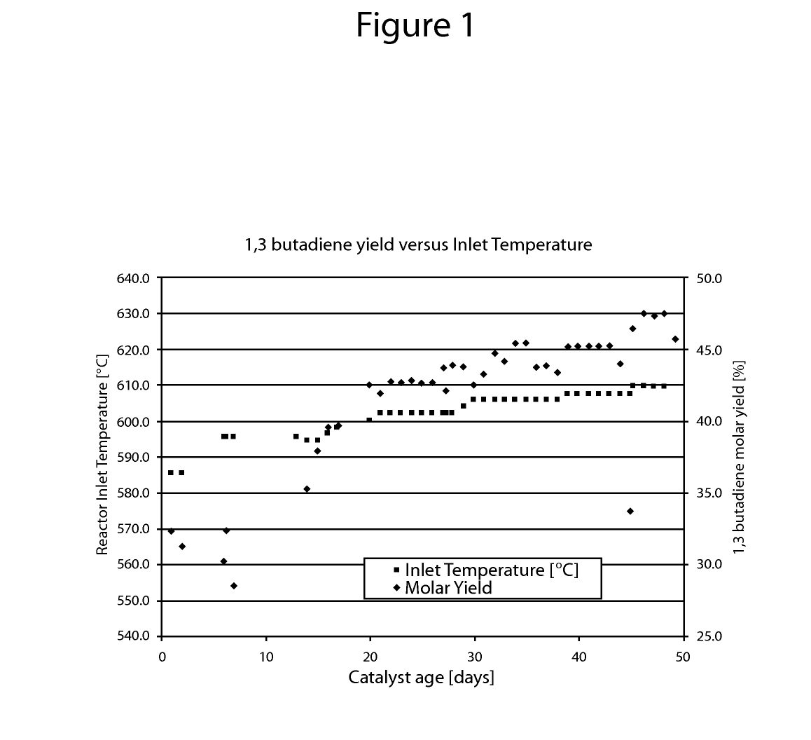

FIG. 1 shows the molar yield of butadiene and reactor inlet temperature over catalyst age. Over the 50 days, 1,3-butadiene molar yield varied from just under 30 mol % to over 45 mol % at temperatures ranging from around 590° C. to 610° C. The yield of butadiene was between 42 mol % and 47 mol % at 602° C. to 610° C. inlet reactor temperature, at 290 mbar pressure, at LHSV of 0.31 hr⁻¹ and a 22:1 steam to hydrocarbon molar ratio.

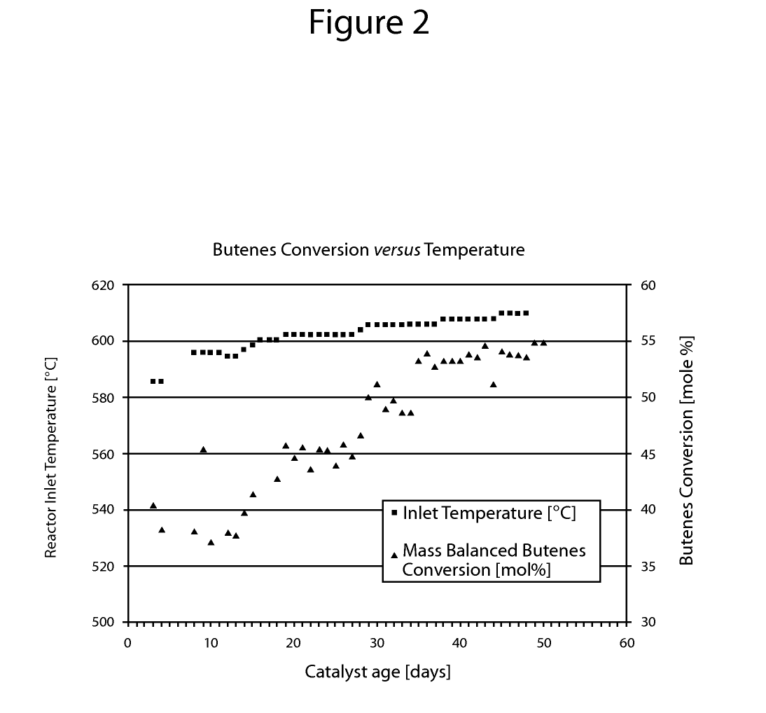

FIG. 2 shows the 1-butene conversion and reactor inlet temperature over catalyst age. Over the 50 days, butene conversion varied from just under 40 mole % to near 55 mole % at temperatures ranging from around 590° C. to 610° C.

Table 1 shows reaction products composition for day 50 on stream. As can be seen in the table, direct dehydrogenation of 1-butene according to the present invention did not lead to significant production of undesirable side products, such as acetylenic compounds. The acetylenic side products were less than 0.1 mol % of the product stream.

TABLE 1
Products composition for 1-butene dehydrogenation.

| Product GAS GC | mol % |
|---|---|
| C5++ | 0.15 |
| Hydrogen | 40.93 |
| Propylene | 1.47 |
| Isobutane | 0.22 |
| n-Butane | 0.15 |
| 1-Butene | 9.00 |
| Isobutylene | 0.00 |
| t-2-Butene | 9.89 |
| c-2-Butene | 6.98 |
| 1,3-Butadiene | 26.21 |
| Isopentane | 0.00 |
| CO₂ | 4.00 |
| Ethylene | 0.10 |
| Methane | 0.90 |
| Total | 100.00 |

Table 2 shows experimental results of dehydrogenation of 1-butene when the steam-to-hydrocarbon ratio (SHR) was varied from 20:1 to 24:1. By increasing the steam to hydrocarbon ratio from 22 to 24, molar yield of butadiene increased. As the SHR was reduced from 24 to 20, molar yield of butadiene dropped to 43.8 mol %.

TABLE 2
Dehydrogenation Conditions: 1,3-Butadiene yield versus SHR

| Catalyst Age (days) | 28 | 29 | 30 | 31 | 32 | 33 | 34 | 35 | 36 |
|---|---|---|---|---|---|---|---|---|---|
| 1-butene (g/hr) PV | 65.2 | 65.2 | 65.2 | 65.2 | 65.2 | 65.2 | 65.13 | 65.13 | 65.13 |
| SHR | 22 | 24 | 24 | 24 | 24 | 24 | 24 | 24 | 20 |
| Pressure mbar | 290 | 290 | 290 | 290 | 290 | 290 | 290 | 290 | 290 |
| Inlet Temp. ° C. | 602 | 604 | 606 | 606 | 606 | 606 | 606 | 606 | 606 |
| Butenes Conversion | 44.8 | 46.7 | 50.1 | 51.2 | 49.0 | 49.8 | 48.6 | 48.6 | 53.3 |
| (mol %) | | | | | | | | | |
| 1,3-Butadiene Yield | 43.9 | 43.8 | 42.5 | 43.3 | 44.8 | 44.2 | 45.5 | 45.5 | 43.8 |
| (mol %) | | | | | | | | | |
| 1,3-Butadiene | 98.1 | 93.8 | 84.9 | 84.5 | 91.4 | 88.9 | 93.5 | 93.5 | 82.2 |
| Selectivity (mol %) | | | | | | | | | |

As used herein the term “conversion” refers to the mol percent of the n-butene content in the feed is converted into product during the dehydrogenation process and is contained in the product stream.

As used herein the term “yield” refers to the moles of butadiene present in the product stream relative to the moles of n-butene converted during the reaction

The term “EB dehydrogenation catalyst” refers to a catalyst based on iron oxide that has the capability to catalyze the dehydrogenation reaction of ethylbenzene to styrene. The EB dehydrogenation catalyst is not limited to a commercially available catalyst or one that is commercially used for the dehydrogenation of ethylbenzene to styrene. The term EB dehydrogenation catalyst would include those catalysts that are in commercial use for the dehydrogenation reaction of ethylbenzene to styrene and catalysts that are commercially available for the dehydrogenation reaction of ethylbenzene to styrene.

The term “catalyst life” refers to the length of time in which a catalyst is active before the catalyst has lost enough catalyst activity to no longer be efficient in a specified process. Such efficiency is determined by individual process parameters.

The term “regeneration” refers to a process for renewing catalyst activity and/or making a catalyst reusable after its activity has reached an unacceptable/inefficient level. Examples of such regeneration may include passing steam over a catalyst bed or burning off carbon residue, for example.

The term “butene” refers to n-butenes or 1-butene, cis-2-butene, trans-2-butene.

The term “butadiene” refers to 1,3-butadiene.

Other possible embodiments include two or more of the above embodiments of the invention. In an embodiment the method includes all of the above embodiments and the various procedures can be carried out in any order.

It is to be understood that while illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the aspects and embodiments disclosed herein are usable and combinable with every other embodiment and/or aspect disclosed herein, and consequently, this disclosure is enabling for any and all combinations of the embodiments and/or aspects disclosed herein. Other and further embodiments, versions and examples of the invention may be devised without departing from the basic scope thereof and the scope thereof is determined by the claims that follow.

What is claimed is:

1. A method for the production of 1,3-butadiene comprising:
contacting an n-butene containing hydrocarbon feedstock with a dehydrogenation catalyst, at a pressure of 1,000 mbar or less, under reaction conditions effective to dehydrogenate said n-butenes to produce a product stream containing 1,3-butadiene at a yield level of at least 40 mol % yield.

2. The method of claim 1, further comprising:
supplying steam to the dehydrogenation reaction in a steam to hydrocarbon molar ratio of at least 10:1.

3. The method of claim 1, further comprising:
operating the dehydrogenation reaction in a reactor at a LHSV of from 0.1 hr⁻¹ to 1.0 hr⁻¹.

4. The method of claim 1, further comprising:
operating the dehydrogenation reaction at pressure of 350 mbar or less.

5. The method of claim 1, further comprising:
operating the dehydrogenation reaction at a temperature of at least 500° C.

6. The method of claim 1, further comprising:
increasing the reactor temperature as needed to keep the butadiene yield at least 40 mol %.

7. The method of claim 1, wherein the yield level of 1,3-butadiene is at least 45 mol %.

8. The method of claim 1, wherein the dehydrogenation catalyst is a commercial dehydrogenation catalyst for the dehydrogenation of ethylbenzene to styrene.

9. The method of claim 1, wherein the dehydrogenation catalyst has an average effective pore diameter of at least 500 nanometers.

10. The method of claim 1, wherein the dehydrogenation catalyst has ferric oxide and potassium as components.

11. The method of claim 1, wherein the product stream contains less than 2 mol % of acetylenic compounds.

12. The method of claim 1, wherein the reaction can operate in excess of 30 days before the catalyst is a deactivated catalyst.

13. The method of claim 1, wherein the reaction can operate in excess of 45 days before the catalyst is a deactivated catalyst.

14. A method for the production of 1,3-butadiene comprising:
contacting an n-butene containing feedstock with a dehydrogenation catalyst, at a pressure of 350 mbar or less, under reaction conditions effective to dehydrogenate at least a portion of said n-benzene to produce 1,3-butadiene;
supplying steam to the dehydrogenation reaction in a steam to hydrocarbon molar ratio of at least 10:1;
operating the dehydrogenation reaction in a reactor at a temperature of at least 550° C.;
increasing the temperature of the reactor as needed to keep the 1,3-butadiene yield at least 40 mol %; and
wherein the reaction can operate in excess of 30 days before the catalyst is a deactivated catalyst.

15. The method of claim 14, wherein the reaction can operate in excess of 45 days before the catalyst is a deactivated catalyst.

16. A method for the production of diolefins in an ethylbenzene dehydrogenation reactor containing a catalyst useful for ethylbenzene to styrene dehydrogenation comprising:
modifying the dehydrogenation reactor to enable the removal of a vapor stream from the reactor and reduce the reactor pressure to vacuum conditions of 500 mbar or less;
supplying a feedstock comprising n-butene to the reactor;
supplying steam to the dehydrogenation reactor in a steam to hydrocarbon molar ratio of at least 10:1;
contacting the olefin containing feedstock and steam with a dehydrogenation catalyst within a reaction zone;
operating the dehydrogenation reactor at a temperature of at least 500° C. and vacuum conditions wherein substantially all of the hydrocarbons are in a vapor phase; and
recovering a vapor product from the dehydrogenation reactor comprising 1,3-butadiene
wherein the vapor product contains less than 0.1 mol % of acetylenic side products.

17. The method of claim 16, wherein the reaction can operate in excess of 30 days before the catalyst is a deactivated catalyst.

18. The method of claim 16, wherein the reaction can operate in excess of 45 days before the catalyst is a deactivated catalyst.

19. The method of claim 16, wherein the yield of 1,3-butadiene is at least 40 mol %.

20. The method of claim 16, wherein the yield of 1,3-butadiene is at least 45 mol %.
